(19) United States
(12) Patent Application Publication
(10) Pub. No.: US 2009/0182324 A1
(43) Pub. Date: Jul. 16, 2009

(54) Laproscopic electronic surgical instruments

(76) Inventor: Mel Kurtulus, La Jolla, CA (US)

Correspondence Address: INTEGRITY IP, P.O. BOX 757, LA JOLLA, CA, 92038, US

(21) Appl. No.: 12/009,174

(22) Filed: Jan. 16, 2008

(51) Int. Cl. A61B 18/12

(52) U.S. Cl. 606/37

(57) ABSTRACT

Surgical instruments are arranged for cutting and cauterizing tissue with a closed-loop wire. An extension/retraction mechanism forces a closed wire loop to constrict upon tissue enclosed by the loop as the loop is retracted and drawn to a smaller size. Simultaneously, as electrical current is passed through the wire loop and further into the tissue, the tissue is resected and cauterized. The closed wire loop is electrically coupled to a remote power source via conductor(s) which cooperate with the extension/retraction mechanisms. The extension/retraction mechanism permits the loop to be extended and retracted as it is adjusted from a first limit position towards a second limit position and respectively back again. Further, the entire instrument is devised to function in cooperation with common laparoscopic port systems. Accordingly, the retraction mechanism permits the closed wire loop to be extended and retracted through a small hollow tube which may be placed in an abdomenal port installed in a surgical process. In the alternative, these devices may be arranged with slight modification to be either bi-polar or monopolar systems.

### BACKGROUND OF THE INVENTIONS

1. Field

The following inventions disclosure is generally concerned with surgical instruments arranged for performing laparoscopic surgeries and specifically concerned with an electrical cutting instrument having a large aperture closed-loop wire with shape memory.

2. Prior Art

A ‘hysterectomy’ is a surgical process to remove reproductive organs of a female—and particularly the uterus. Common means of effecting hysterectomy may typically include an open abdominal process. Open abdominal surgery permits a surgeon ready access and clear view of the organs in question. However, significant increased risk of injury and infection is assumed as it is necessary to cut large incisions in the abdominal wall to sufficiently reach, manipulate and remove organ(s).

One alternative exists where the vaginal canal is used to reach the uterus, but removing the uterus through the vagina requires also removing the cervix which is not always desirable. It is generally preferred that as little tissue as possible be removed and it is excepted that where possible it is best to not disturb the cervix. Accordingly, the procedure has serious disadvantage in some cases.

While a hysterectomy may be achieved via many different procedures, laparoscopic hysterectomy is preferred for its safety, brevity, and reduced recovery time, reduces blood loss, low risk of complications, among others. Modern medical science permits removal of a uterus through very tiny incisions in the abdomen. Among various versions of laparoscopic hysterectomy procedures are those in which an electrical cutting knife and cauterizing tool are used to separate or resect tissue and organs from where they are attached.

In general, an electrode tipped instrument severs the uterus from the cervix at the isthmus. In some cases, the ovaries are also cut away from where they are attached at the uterus. In other cases, the ovaries are separated from their attachment to the body and are removed along with the uterus. To separate tissue in this way, a surgeon uses the tip of an electrical knife to apply electrical current to the region of tissue where resection is desired.

In most conventional instruments, the electrical knife is arranged as a monopolar system having a single electrode at the instrument tip. A patient's body or bulk tissue forms an electrically conductive path to a second electrode of opposing pole (sometimes referred to as a ‘ground’ or ‘earth’). Current density is very high at the knife edge, but very low at the grounding electrode. Accordingly, cutting and cauterization only occurs at the tip of the instrument with very little or negligible damage to tissues near the other electrode which is spread over a considerably large area.

While monopolar systems are considered safe, it remains desirable to avoid unnecessarily passing electrical currents through healthy tissue. Where it is possible, a second electrode should be placed where it can receive the electrical current just after it passes through and cuts tissue at the site where it is applied. Bipolar systems are sometimes arranged with both electrodes incorporated in the cutting instruments tip. In this way, electrical current passes from the first electrode, briefly through a small portion of tissue, and thereafter returns at the electrode of opposite pole. The electrical current only interacts with a very small volume of tissue, and specifically with the portion of tissue intended to be manipulated (cut or cauterized). Because of this, bipolar systems are sometimes preferred as more efficient and safe. However it is not always easy to configure to electrodes in a shape such that the action of current corresponds to the nature (shape, size, et cetera) of the tissue to be manipulated. That is, sometimes the spatial nature of the tissue does not cooperate well with the electrode shapes which might be available at a tool tip. Constraints on current density further complicate possible tool configurations. To provide two electrodes (for bipolar instruments) in a spatially advantageous arrangement which cooperates with the shape and sizes of organs or tissues being addressed is the essence of effective instrument design.

Modern surgical technologies permit operations including removal of large organs through very small abdominal incisions in processes known as ‘laparoscopic surgery’. In a laparoscopic surgery, a plurality of small abdominal incisions are made and prepared with special port systems to permit elongated instruments not larger than about 10 mm to about 15 mm in cross-section to be inserted into the abdominal cavity. Video cameras, cutting instruments, grabbing tools, among others are inserted via these abdominal ports and a surgeon manipulates them to operate upon various organs and tissues. After organs are removed via a laparoscopic surgery, a patient quickly and easily heals as there is considerably less damage to the abdominal wall. Further, operating times are often reduced thus improving efficiency.

Part of the laparoscopic supracervical hysterectomy includes cutting the uterus away from the fallopian tubes and its blood supply. After it is clear of these, it must be severed from its remaining attachment at the cervix. In most common laparoscopic processes, and an electrical knife is used to resect first on one side, and then on the other to finally separate the uterus from the cervix. As the cutting and manipulation of the organ is done via a video camera view of the abdominal interior, the process of severing the uterus from the body can take a considerable amount of time. It is a difficult process and takes very good concentration from a well trained and experienced surgeon to do well and safely. Even where a highly skilled surgeon in best conditions applies this process, the resection takes a considerable amount of time and the cut may be less than perfectly ‘smooth’ or ‘clean’ as it is generally made via a series of individual cuts.

As such, certain inventions are now being introduced to improve a doctor's ability to cut a uterus from the cervix. In one important system, sold commercially by “Medical Dynamics” known by its trade name: “Lap Loop”, is described in the Journal of Minimally Invasive Gynecology, Vol. 12, No. 4, July/August 2005. An open loop wire is guided around the fundus portion of the uterus by a specially shaped ‘introducer’. After the ‘cable’ is routed properly, the loop is closed by fastening the cable ends to special hardware provided for such. The instrument includes this hardware to couple a length of cable with specially prepared ends that cooperate with a socket of the instrument. Because of the nature of the hardware, there is no electrical isolation between various elements and as such the device is restricted to monopolar arrangements. Application of current causes the uterus to be sectioned clean in a single plane leaving a preferred, tidy cut. Further, the resection step is quick and smooth. However, this instrument involves considerable effort to first properly thread the cable around the uterus, and still further to couple the cable ends to the tool sockets in order to realize a closed-loop before application of electrical current.

While systems and inventions of the art are designed to achieve particular goals and objectives, some of those being no less than remarkable, these inventions have limitations which prevent their use in new ways now possible. Inventions of the art are not used and cannot be used to realize the advantages and objectives of the inventions taught herefollowing.

It should be understood that all of herein referenced materials provide considerable definition with regard to elements of these inventions. Therefore, those materials are incorporated herein by reference whereby this specification can rely upon them for enablement of the particular teachings of each.

### SUMMARY OF THESE INVENTIONS

Comes now, Mel Kurtulus with inventions of laparoscopic surgical instruments having closed-loop arrangements for improved resection efficiency including devices and methods of use of same. It is a primary function of this these devices and methods to provide efficient means of resecting human organs via laparoscopic facility and systems. A fundamental difference between the instant invention and those of the art can be found when considering its closed-loop nature, and in some preferred versions its bi-polar arrangements.

Laparoscopic electronic surgical instruments are devised in which a closed-loop wire portion having shape memory (spring wire) and additional facility to cause the closed-loop take shape and size which cooperates with a human uterus. As such, these instruments provide a loop which is easily passed over the uterus when the instrument is in an extended state. Once passed over the uterine fundus and properly fitted and constricted about an isthmus portion of the tissue between the uterus and cervix, an electrical current may be applied to effect a most efficient resection with accompanied cauterization. The closed-loop wire made thereafter be fully retracted and the entire instrument removed from the abdomen via common “trocars” or abdominal ports used in conjunction with other laparoscopic instruments. Among two of the most preferred versions include: a bipolar version and a monopolar version.

A bipolar version, a single closed-loop is formed of two electrode portions joined together at an electrical isolator. Each of the electrodes may be coupled to the opposing poles of an electrical current source whereby current passing between them tends to cut and cauterized tissue in close proximity. Specifically, when the closed-loop is constricted down upon a cylindrical body of tissue, electrodes arranged in this fashion tend to optimally inject electrical current in a preferred spatial sense into tissue to cause most efficient cutting action.

In a monopolar version, a single closed-loop is formed of one electrode coupled to an electrical source. A cooperating ‘collector’ electrode may be established at another portion of the patient's body. When the closed-loop in the monopolar configuration is closed down upon and constricted about a cylindrical body of tissue, the loop injects electrical current in a manner to bring about efficient resection.

In both cases, the instrument is arranged such that further constriction of the closed-loop is applied while current is passed to still better couple the devices electrode(s) to tissue yet to be cut/cauterized. Further, these instruments are specifically arranged to cooperate with a standard laparoscopic supporting apparatus. In particular, they are elongated and quite narrow in cross section so that they might be used with abdominal ports which permit access to the interior of a body cavity via small incisions therein.

### OBJECTIVES OF THESE INVENTIONS

It is a primary object of these inventions to provide new laparoscopic surgical instruments

It is an object of these inventions to provide electrical resection instruments for laparoscopic use.

It is a further object to provide electrical resection instruments having configurations which cooperate with a human uterus.

It is an object of these inventions to provide electronic resection instruments having a closed-loop system which facilitates coupling with respect to a human uterus.

A better understanding can be had with reference to detailed description of preferred embodiments and with reference to appended drawings. Embodiments presented are particular ways to realize these inventions and are not inclusive of all ways possible. Therefore, there may exist embodiments that do not deviate from the spirit and scope of this disclosure as set forth by appended claims, but do not appear here as specific examples. It will be appreciated that a great plurality of alternative versions are possible.

### BRIEF DESCRIPTION OF THE DRAWING FIGURES

These and other features, aspects, and advantages of the present inventions will become better understood with regard to the following description, appended claims and drawings where:

FIG. 1 is a cross sectional diagram of a closed-loop laparoscopic instrument of these systems;

FIG. 2 is another cross sectional diagram (of a tip portion of one version of these instruments) with expansion in one dimension for additional clarity;

FIG. 3 illustrates a tip portion of one version where the instrument is in a retracted position as opposed to the contrasting extended position illustrated in FIG. 2;

FIG. 4 illustrates an important preferred bi-polar version of these systems; and

FIG. 5 illustrates an important preferred monopolar version of these systems.

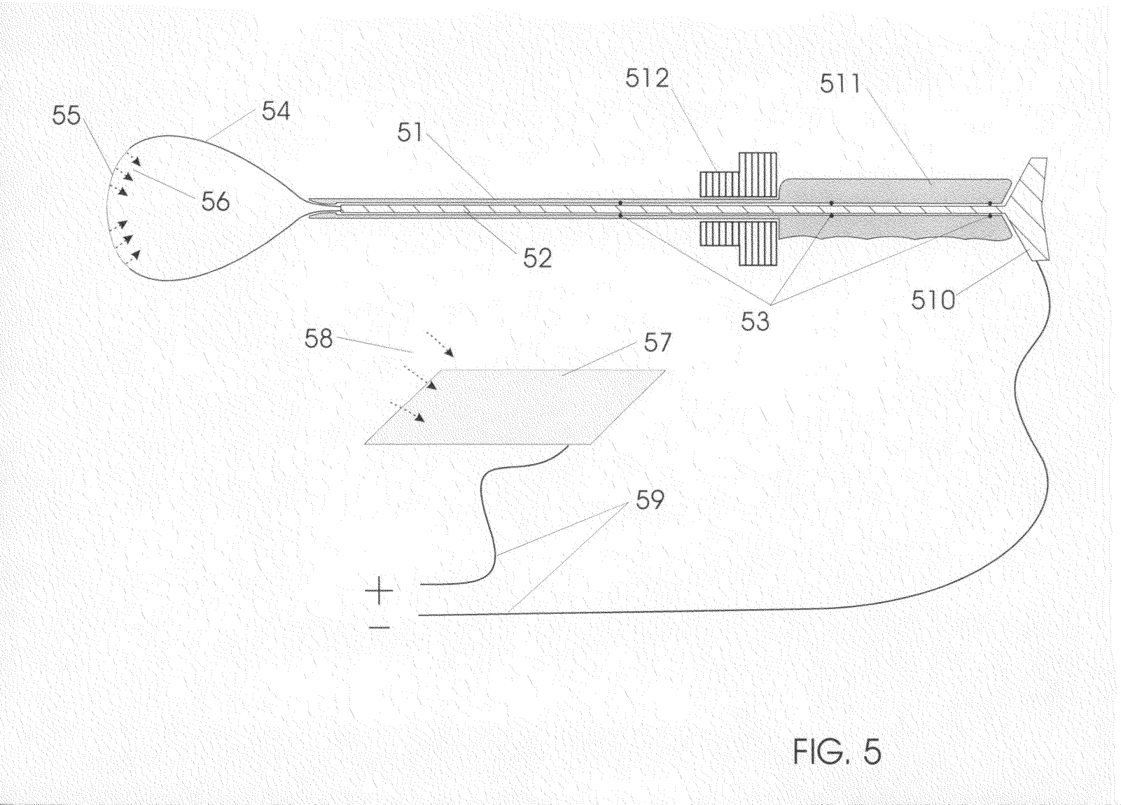

### PREFERRED EMBODIMENTS OF THESE INVENTIONS

In accordance with each of preferred embodiments of the invention laparoscopic surgical instruments having closed-loop arrangements are provided. It will be appreciated that each of the embodiments described include an apparatus and that the apparatus of one preferred embodiment may be different than the apparatus of another embodiment. Accordingly, limitations read in one example should not be carried forward and implicitly assumed to be part of an alternative example.

A laparoscopic instrument for use in hysterectomy surgeries is fashioned with an electrode or electrodes which cooperate with the particular size, nature, and characteristics of human organs and in particular the uterus, ovaries, and cervix. Further, these instruments are particularly arranged with structures which cooperate with the physical nature of common laparoscopic dimensions; including common sized abdominal ports or abdominal volume, et cetera. An electrode or electrodes formed of an electrically conductive materials such as metal and more preferably spring wire arranged in a closed-loop sufficient in size to be passed over a large uterus includes the active portion of a laparoscopic resection instrument for cutting and cauterization.

The closed wire loop is further arranged to cooperate with a shaft member and a tubular member such that the wire loop is retractable and extendable in conjunction with linear displacements between the shaft and tube. Because a wire loop retracted into a narrow tube is very thin, the arrangement is ideal for use in laparoscopic type systems where small abdominal ports provide access to the inside of an abdominal cavity. In some most important versions, a closed wire loop is formed of two electrically isolated portions thus forming a bipolar system enabling unique current flow patterns particularly advantageous for cutting tissues of a specific shape. In particular, where a wire loop is constricted about a cylindrical body, a cross-sectional cut may be effected cleanly and with ease. Most importantly, a closed-loop spring wire system in a bipolar arrangement provides an electric current flow pattern having good spatial relationship with the tissue to be resected.

Once a closed-loop wire as described is passed over a uterus and placed near where the uterus meets the cervix, the loop is pulled into the tubular member such that the loop portion remaining outside of the tube is constricted further down upon the tissue which it surrounds. Once the wire is pulled into the tube as far as the tissue allows, a current may be applied in a manner to resect the tissue in contact with the wire. As the tissue is cut, the loop may be further retracted into the tube and further constricted down upon the remaining uncut tissue.

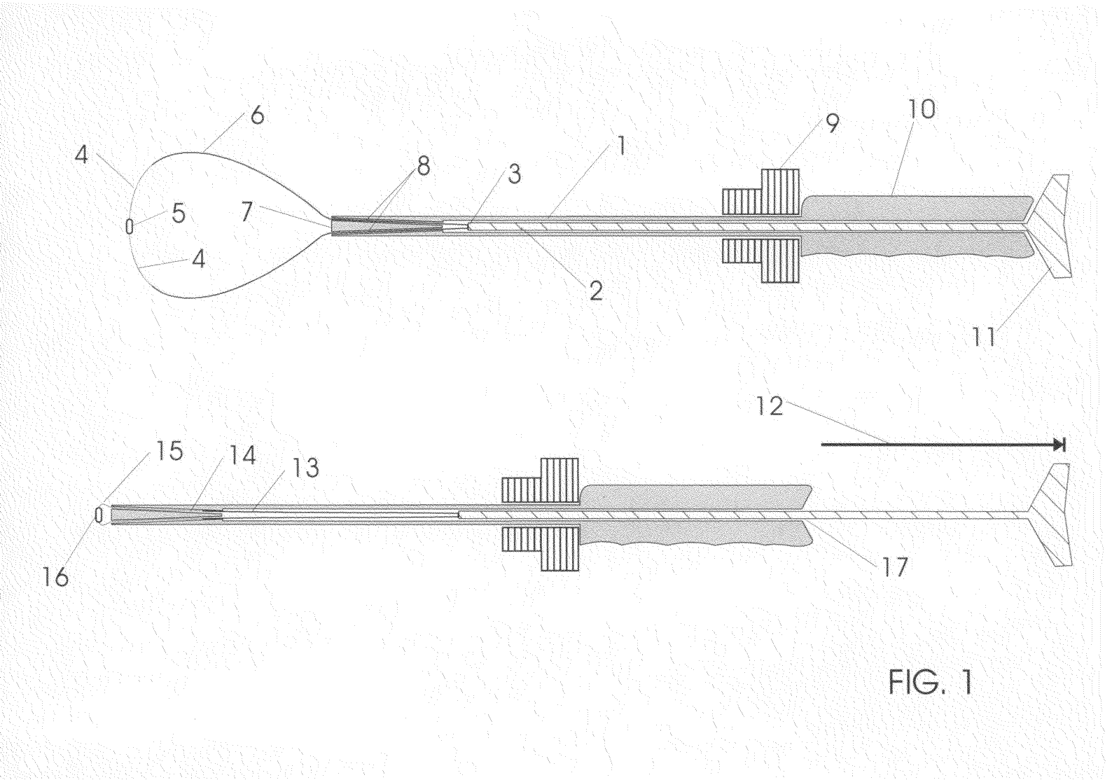

The aspect ratio of these instruments is quite large and usually greater than about 10:1. As such, the drawing figures cannot easily be drawn to proper scale in both dimensions on sheets of acceptable size. It should be appreciated that scale is not meant to be accurately depicted in these drawing figures which are useful for illustration purposes by not engineering. For clarity, one dimension may be greatly expanded in relation to an orthogonal dimension. While FIG. 1 is presented with only slight distortion in this regard, in the interest of clarity the other figures are expanded in the transverse dimension with respect to the instruments longitudinal axis. With reference to FIG. 1, a surgical instrument suitable for use in laparoscopic operations is depicted in two views including a first extended view, and a second retracted view. “Extended” and “retracted” are with reference to the active portion of the instrument, a closed wire loop.

The primary elements of the device include: an elongated tubular member 1, a cooperating shaft member 2 of similar size and aspect ratio, and a closed wire loop system. The closed wire loop system is coupled thereto and affixed at the tip end 3 of the shaft member. The shaft member is arranged whereby it shares a longitudinal axis with the tubular member and moves slidably therewithin a portion of that tubular member.

The closed wire loop system may be comprised of a plurality of discrete portions. In some most preferred versions, a closed wire loop is comprised of two electrically isolated conductor elements 4, a coupler element 5 which is electrically insulative and mechanically binds the two conductor elements at a junction small in size. An insulative outer coating 6 covers and electrically isolates either conductor which may run through the center of the outer coating. Metallic portions of the wire loop may be in some versions formed of metallic spring wire having a “memory” shape and size which cooperates in particular with select human organs; for example a uterus. When the closed-loop is in an extended state, the spring wire encourages the closed-loop to take a prescribed shape in accordance with the spring design imparted during its manufacture. Normal use of the instrument, includes passing the closed-loop over and around a uterus. Accordingly, it is of great advantage if the size and shape of the loop approximates or slightly exceeds that of the outside diameter of a large human uterus, thus facilitating passing the loop over the organ. It is for this reason that an instrument in a fully extended state includes a loop of shape and size brought about via the spring action of the metallic conductor, to cooperate with the objective of passing the loop over a uterus.

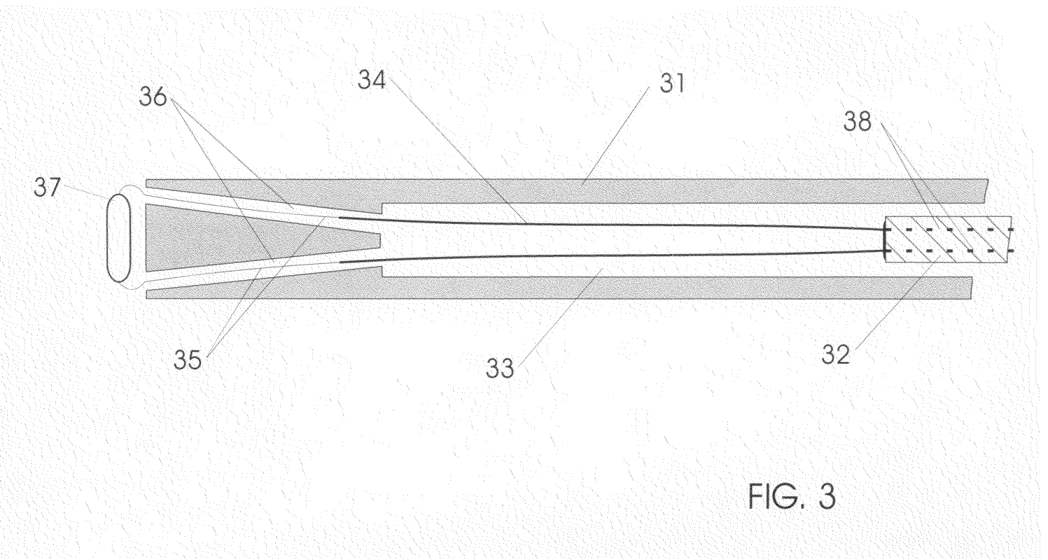

The coupling between the closed wire loop and the shaft tip end permits the loop to be mechanically affixed thereto such that linear motion of the shaft imparts a translation force on the closed wire loop which tends to either retract or extend the loop with respect to the tubular member. The shaft and wire loop move together as one within the tubular member. Further, the coupling between the closed wire loop and the shaft is arranged to permit extension of electrical conductor(s) through the shaft body such that electrical current may be coupled from a remote source to the closed wire loop. This is clarified in further detail in FIG. 3.

The closed wire loop further may be related to the tubular member in that the tubular member may include a shaped tip 7 which imparts a slight expanding force which encourages the loop to open as the loop is pushed from the tubular interior to the extended state. Thus the loop slightly expands therefrom and retracts thereinto under motivation of the shaft which moves slidably within the tubular member.

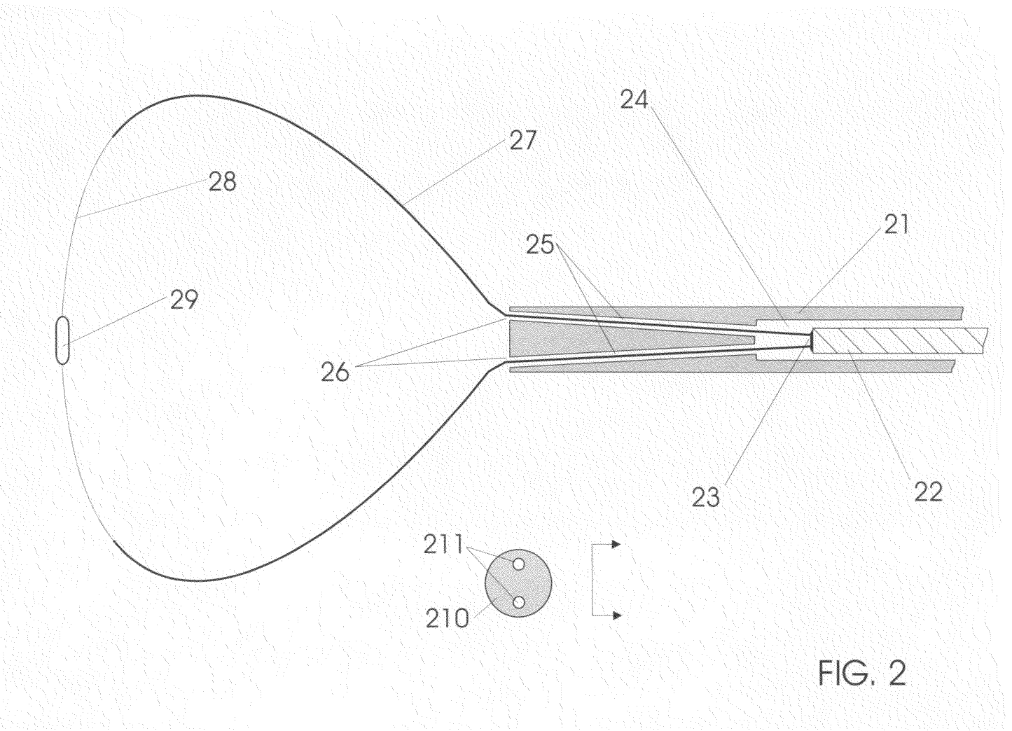

In some preferred versions, a specially arranged end of the tubular member provides electrical isolation between two conductors as it provides spatial isolation therebetween. A pair of channels 8 or elongated holes are provided in a solid electrically insulating material such as a plastic or ceramic and each of two discrete exposed wire portions of the closed wire loop pass through these channels whenever the closed-loop is brought into a fully retracted position. It is best if the extent of these channels is slightly longer than the length of the exposed conductor on either side to assure that electrical isolation is provided when the closed wire loop is fully retracted. Close-up and detailed FIGS. 2 and 3 showed this more clearly.

An ‘extended state’ of the instrument represented in the top half portion of FIG. 1 where the closed-loop which is large and open and extends outwardly with respect to the tubular member. A ‘retracted state’ of the instrument is represented in the bottom half portion of FIG. 1. where the closed-loop has been pulled into the tubular member. A linear translation 12 between the tubular member and the shaft member causes the shaft tip to be pulled further into a cavity within the tubular member and accordingly causes the closed wire loop 13 to simultaneously be pulled thereinto. When fully retracted, a portion 14 of the closed wire loop remains in the channels and a portion 15 along with the coupler element 16 remains slightly external but tightly positioned at the tubular member tip/orifice(s). The shaft member extends from the tubular member at an exit orifice 17.

The arrangement is more readily understood in view of the drawing of FIG. 2 which shows an expanded view along with a cross-sectional view of the tubular member end with two orifices. The tubular member 21 (shown only at a distal end) has axially therein shaft member tip 22 where closed wire loop is affixed at shaft tip 23 whereby displacement forces on the shaft are transmitted to the closed wire loop. In some best versions, the tubular member is further characterized as having an internal cavity 24 which may be shaped to provide a mechanical limit or “stop” whereby the shaft is prevented from further displacement in the direction of the tubular end. Channels (two) 25 formed into the solid electrical insulator material of the tubular member provide orifices 26 from which portions of the closed wire loop may pass. In one example, a majority portion 27 of the closed wire loop may be comprised of an electrical spring wire conductor with an insulating outer coating. An exposed portion of the wire connector 28 (also spring wire) has no insulator covering and promotes the possibility of having electrical current emit therefrom. A ceramic or plastic insulator “bead”, or coupler element 29 mechanically connects the two wire portions while simultaneously maintaining electrical isolation therebetween. The coupler element may provide a mechanical bond via a mechanical interlocking system, alternatively via adhesives, or other bond system which ensures a strong mechanical connection and electrical isolation. In cross sectional view, the tubular member end 210 includes two orifices 211 through which the closed-loop wire portions may be passed.

In a retracted state, the instrument is described and illustrated in the diagram of FIG. 3. Tubular member 31 has shaft number 32 therein axially in cooperation therewith such that the shaft may be displaced in a sliding action along the axis shared by each. Tubular member cavity 33 may contain therein a portion of retracted closed wire loop; more particularly conductor with insulated outer coating 34. The outer coating may be preferably made of a plastic material which forms a low coefficient of friction with respect to the material from which the tubular member is constructed. In this way, the relationship between the closed wire loop and the channels of the tubular member is improved as the wire loop will more easily slide from a retracted to extended state and reverse. Silicon material is one example which provides an electrical insulation function while at the same time serves to provide a sliding system. Official notice is taken here that many materials will be suitable for a similar function. Bare (metallic) wire loop portions 35 do not include an insulator coating. This portion of the closed wire loop is operable to emit electrical current therefrom such that electrical current passes into/through tissue to be cut or cauterized. However, when the instrument is in a retracted state as shown in the figure, the bare wire portion is protected against electrical shorts as they are contained in the channels 36 of the tubular member end.

Coupler element 37 provides a mechanical bond between the two wire portions and electrical isolation therebetween. This coupler element additionally provides a mechanical stop against pulling the shaft element too far down the axis of the tubular element. When the closed wire loop is fully retracted, the coupler element comes to a rest at the tubular element tip and prevents further displacement between the shaft element and the tubular element. Finally, electrical conductors 38 shown in phantom pass from the closed wire loop where they are in electrical contact therewith, through the shaft system to exit at the instrument's other end where they may extend further to be energized by a remote power supply. Electricity from a power supply outside the abdomen passes to a wire loop inside an abdomen via the shaft and tubular member which operate in cooperation with a trocar to provide access to the uterus.

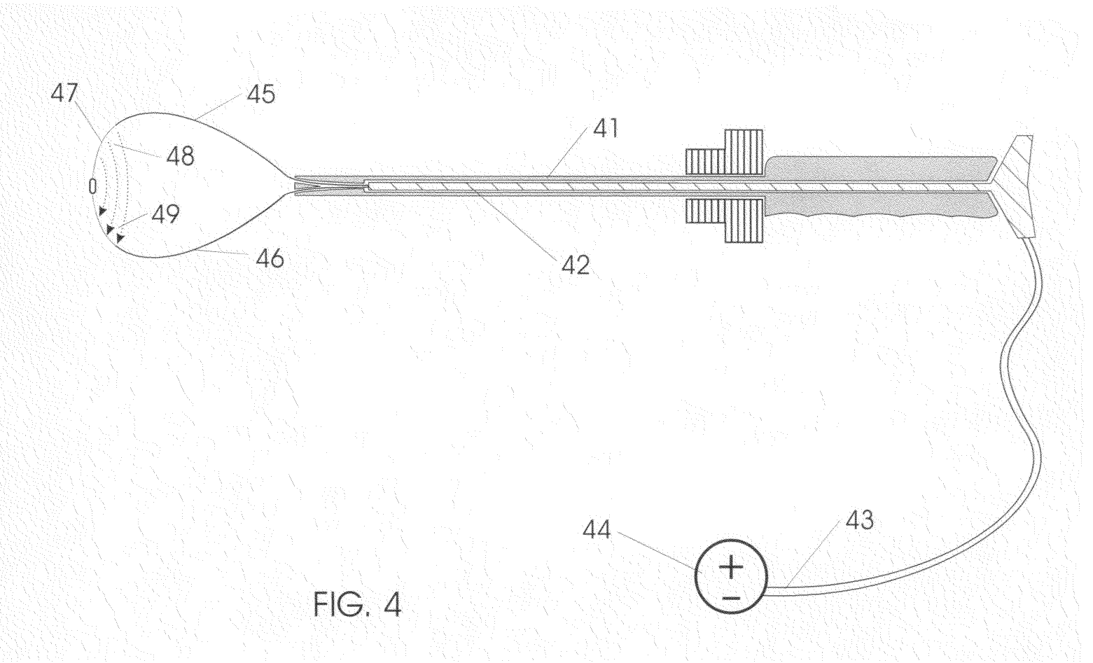

FIG. 4 illustrates a most important version of these laparoscopic instruments. The device presented in the figure is a bipolar version of the device. An elongated tubular member 41 shares an axis with shaft member 42. Two electrical leads 43 extend from the end of the instrument and receive electrical power from power supply 44 which may be switched by a surgeon and is arranged to provide either DC, AC, or combinations of DC and AC electrical currents. An insulated electrical lead of first polarity 45 carries electrical current to closed wire loop portion 47 (non-insulative) where it leaves 48 the conductor to pass through tissue. After passing through tissue where the tissue may be cut and/or cauterized, the current is collected 49 at the complementary portion of the wire loop—at the exposed conductor of opposite polarity. Insulated electrical lead 46 is a return path for electrical current which passes through the shaft back to the supply. It is in this manner that a bipolar instrument is preferably realized. A bi-polar instrument in accordance with these teachings provides an excellent current density distribution quite suitable for resecting tissue. This is especially the case where the tissue being resected is generally cylindrical in nature such as the portion of the uterus near where it joins the cervix. That is, the cutting action of the instrument is particularly designed to cooperate with the particular anatomy to which it is intended to be applied. The length of both exposed portions of the conductors is chosen in view of the average size of this portion of the human organs. In some cases, this size may be adjusted to produce an instrument more suitable for larger or smaller persons. In preferred versions, these conductors are between about 10 mm and 50 mm.

While in some versions, a bipolar closed wire loop tool as described is most efficient, it remains clear that other versions of these instruments, i.e. those arranged in monopolar configurations also provide excellent benefit. Monopolar versions are accompanied by added simplicity as only one electrical lead is necessarily provided to carry current through the shaft member to the closed wire loop. Tubular member 51 supports therein shaft member 52 which might include the coupling of which further improves an airtight seal of nicely fitted “ο” rings 53. Laparoscopic operations sometimes used gas to inflate the abdominal cavity, as such it is sometimes desirable in laparoscopic instruments to provide for mechanisms to reduce loss of gas from the abdomen.

A single conductor 54 forms the closed wire loop and includes a covered portion and an exposed (no insulation) portion 55 from which electrical current 56 may pass into surrounding tissue. In a monopolar system, electrical current may be collected at a receiving conductor 57 coupled to a large area of a patient's skin surface. Current from the instrument passes through tissue and returns 58 to the electrical source via the conductor. Since the conductor is coupled over a large area, the current density is greatly reduced for all portions of tissue in which current passes except for the tissue nearest the wire emitter (closed wire loop). Cutting and cauterizing therefore only occurs at the wire loop where the current density is highest. Electrical leads 59 include a first lead of first polarity and a second lead of opposing polarity where only one of these is necessarily passed through the instrument's shaft element whereafter it is coupled at the closed wire loop. Similar to the closed wire loop of the bi-polar instrument, when the loop is constricted upon tissue, the exposed portion of the conductor is well suited with respect to size when compared to the site where it is desired that resection occurs.

To improve handling of these devices by the surgeons who use them, both shaft member and tubular member may be terminated on one end by ergonomically shaped handles; i.e. a shaft handle 510 and tube handle 511. Abdominal port element 512 which may be a standard device well known and used with a great plurality of laparoscopic devices couples these instruments to a patient's abdominal cavity in a conventional way.

One will now fully appreciate how high performance surgical instruments for laparoscopic hysterectomy may be realized as closed-loop systems and the advantages thereof. Although the present invention has been described in considerable detail with clear and concise language and with reference to certain preferred versions thereof including best modes anticipated by the inventors, other versions are possible. Therefore, the spirit and scope of the invention should not be limited by the description of the preferred versions contained therein, but rather by the claims appended hereto.

1) Surgical instruments for use in laparoscopic procedures comprising: an elongated tubular member;
an elongated shaft member slidably disposed coaxially within said tubular member whereby linear displacements between the two are enabled;
a closed wire loop affixed at one end of the shaft member; and
a power supply electrically coupled to said wire loop the whereby electrical current is passed from a first portion of the loop to tissues thereby cutting and/or cauterizing the tissue.

2) Surgical instruments of claim 1, said closed wire loop is arranged with two electrically isolated electrodes of opposing pole.

3) Surgical instruments of claim 2, said wire loop is further comprised of at least two electrical conductor elements (wire) of opposing pole separated by an insulating coupler element.

4) Surgical instruments of claim 3, said insulating coupler element further characterized as an electrical insulator forming a bond at an end of each electrical conductor element.

5) Surgical instruments of claim 3, said “closed wire loop affixed at one end of the shaft member” being further characterized as a coupling to which each [from which the two electrical conductors extend and separate spatially] of the two electrical conductors is affixed, the coupling permitting the conductors to extend from the shaft tip while maintaining electrical isolation from each other.

6) Surgical instruments of claim 5, said shaft further comprising at least one electrical conductor connecting to the closed loop and further passing from an exit orifice of said tubular member.

7) Surgical instruments of claim 5, said tubular member further comprising a tip with a mechanism for encouraging separation of wire loop portions as a shaft is slid from a retracted position to an extended position.

8) Surgical instruments of claim 1, closed wire loop extends from at least one aperture in said tubular member and is driven from a retracted position to an extended position and visa-versa in response to displacements between said shaft member and said tubular member.

9) Surgical instruments of claim 8, said tubular member is formed to include a mechanical stop which prevents the tip end of shaft from extending past a prescribed limit position with respect to the tubular member.

10) Surgical instruments of claim 1, said tubular member and shaft member have tactile grip elements which facilitate ergonomic grasp by a surgeon's hands whereby they may be manipulated to yield a displacement therebetween the tubular member and the shaft member.
